(19) United States
(12) Patent Application Publication
(10) Pub. No.: US 2014/0056834 A1
(43) Pub. Date: Feb. 27, 2014

(54) COSMETIC COMPOSITION COMPRISING CHRYSOPHANOL AS ACTIVE INGREDIENT FOR SKIN WHITENING

(71) Applicant: PUKYONG NATIONAL UNIVERSITY INDUSTRY-UNIVERSITY C, Busan (KR)

(72) Inventors: Se-Kwon KIM, Busan (KR); Wijesekara Liyanae Isuru WIJESEKARA, Busan (KR)

(73) Assignee: Pukyong National University Industry-University Cooperation Foundation, Busan (KR)

(21) Appl. No.: 13/665,454

(22) Filed: Oct. 31, 2012

(30) Foreign Application Priority Data
Aug. 23, 2012 (KR) 10-2012-0092472

(51) Int. Cl. A61K 8/35; A61Q 19/02

(52) U.S. Cl. CPC A61K 8/355; A61Q 19/02
USPC 424/62; 552/262

(57) ABSTRACT

A novel use of chrysophanol for skin whitening is provided, in which chrysophanol is included in a cosmetic composition as an effective ingredient for skin whitening. The chrysophanol compound has excellent stability and can also effectively inhibit melanin production via mechanism of inhibiting expression and activity of tyrosinase.

### CROSS-REFERENCE TO RELATED APPLICATIONS

This patent application claims the benefit of priority from Korean Patent Applications No. 10-2012-0092472 filed on Aug. 23, 2012, in the Korean Intellectual Property Office, the disclosure of which is incorporated herein by reference in its entirety

### BACKGROUND OF THE INVENTION

1. Field of the Invention

The present invention relates to a novel use of chrysophanol for skin whitening, and more particularly, to a cosmetic composition for skin whitening, which comprises chrysophanol as an active ingredient.

2. Description of the Related Art

Skin is very important organ that has biochemical and physical functions, because it protects a human body while directly contacting exterior environment. The skin is mainly divided into epidermis, dermis, and subcutis. Human skin has varying colors depending on the number, size, type and distribution of melanin-containing melanosomes which are present mainly in skin cells.

Melanosomes are produced by melanin cells, and dark-pigmented melanin is produced by melanocyte. Melanin absorbs light energy of ultraviolet rays in the sunlight to thus prevent the cells located therebelow from being damaged by the ultraviolet rays. Abnormal deficiency in the production of melanin causes abnormal skin condition known as albinism, while excessive synthesis of melanin due to ultraviolet rays or the like causes skin damage, freckles, or even skin cancer in severe cases.

Tyrosinase plays an important role in the formation of melanin. This particular enzyme generates dopaquinone acting on amino acid (“tyrosine”) present in tissues. The black pigment melanin is produced from dopaquinone after a series of auto-oxidations. Given this, a composition for skin whitening can be produced in various manners, by inhibiting melanin production in specific process based on the following.

First, a component that can block ultraviolet rays, which is the main cause of melanin production, may be used. A composition according to the above-mentioned method contains light scattering or blocking agent to be able to reduce DNA damage or inflammation due to ultraviolet rays, thus inhibiting synthesis of core carbohydrate which is necessary for the activity of tyrosinase such as glucosamine and subsequently inhibiting production of melanin (Applicable in whitening stage A). Second, a substance such as enzyme acid (kojic acid), leucinol, uric acid or arbutin which hinders function of tyrosinase involved in the melanin production can be used (Applicable in whitening stage B). Third, dopachrome is stabilized to inhibit development into eu-melanin, the black pigment melanin (Applicable in whitening stage C). Fourth, substance such as vitamin C can be used, which provides strong anti-oxidation effect to thus bring in whitening effect, in the process that tyrosine is converted to dopa and dopachrome and undergoes complicated oxidation and condensation reactions (Applicable in whitening stage D). Fifth, enzyme or alpha hydroxyl acid (AHA) can be used to remove and reduce the produced melanin from horny layer (Applicable in whitening stage E).

In the cosmetics industry, substances such as kojic acid or arbutin that inhibit enzymatic activity of tirosinase, hydroquinone, L-ascorbic acid and derivatives thereof and various plant extracts have been conventionally used as the elements that confer whitening effect. However, because the substances that inhibit enzymatic activity of tyrosinase have low stability among the prescriptions, the use thereof is limited for concerns such as decomposition and discoloration, generation of unfavorable odor, unsure efficacy or effect in biological level, and safety.

Accordingly, in a continuous effort to find a substance that is derived from natural product and thus is stable in human body, and can also provide excellent whitening effect, the present inventors focused on a chrysophanol compound isolated from marine-derived fungus, Microsporum sp. and identified, and as a result of testing whitening effect, could confirm that the chrysophanol compound can dose-dependently inhibit the expression and activity of tyrosinase and thereby effectively inhibit synthesis of melanin.

### SUMMARY OF THE INVENTION

An objective of the present invention is to provide a cosmetic composition which is stable in a human body and also provides excellent melanin-inhibiting activity, and thus can provide effective skin whitening effect.

In order to achieve the objective explained above, an embodiment provides a cosmetic composition comprising chrysophanol or a pharmaceutically-acceptable salt thereof as an effective ingredient, for skin whitening.

In one embodiment, the chrysophanol may be derived from marine-derived fungus, Microsporum sp.

In one embodiment, the chrysophanol may inhibit production of melanin via mechanism of inhibiting expression and activity of tyrosinase.

In one embodiment, the chrysophanol may be included in the composition in a concentration of 10 to 50 μM.

In one embodiment, the composition may be formulated into one selected from the group consisting of: skin lotion, skin softener, skin toner, astringent, lotion, milk lotion, moisture lotion, nutrition lotion, massage cream, nutrition cream, moisture cream, hand cream, essence, nutrition essence, pack, soap, shampoo, cleansing foam, cleansing lotion, cleansing cream, body lotion, body cleanser, serum, lipstick, makeup base, foundation, pressed powder, and loose powder.

Because the chrysophanol compound according to embodiments can effectively inhibit production of melanin based on excellent stability and also a mechanism of inhibiting expression and activity of tyrosinase, a composition comprising the chrysophanol compound as an effective ingredient provides excellent whitening effect based on melanin pigment inhibition without irritating skin, and thus can be advantageously used as a functional cosmetic composition.

### BRIEF DESCRIPTION OF THE DRAWINGS

The foregoing and/or other aspects of the present invention will be more apparent upon reading the description of certain exemplary embodiments with reference to the accompanying drawings, in which:

FIG. 1 shows morphological character and appearance of marine-derived fungus, Microsporum sp., isolated from the surface of marine red alga, Lomentaria catenata;

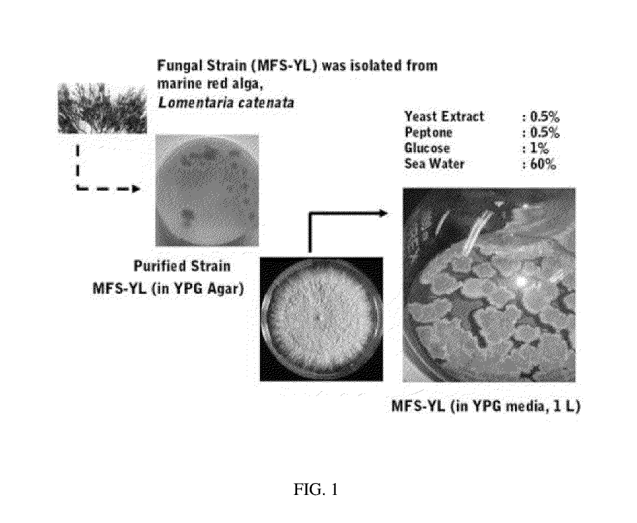

FIG. 2 schematically shows a process of obtaining ethylacetate extract of MFS-YL fungal strain of marine-derived fungus, Microsporum sp.;

FIG. 3 schematically shows a process of isolating chrysophanol compound from MFS-YL fungal strain of marine-derived fungus, Microsporum sp.;

FIG. 4 is 1H-NMR (a) and 13C-NMR (b) spectra of chrysophanol;

FIG. 5 is a graph representing cell viability of chyrsophanol in B16F1 melanoma cells in dose-dependent manner;

FIG. 6 shows measurement of tyrosinase inhibitory activity of chrysophanol using mushroom tyrosinase assay;

FIG. 7 shows measurement of inhibition level of melanin formation of chrysophanol in B16F1 melanoma cells in dose-dependent manner;

FIG. 8 is a graph representing measurement of melanin content in B16F1 melanoma cells by chrysophanol in dose-dependent manner;

FIG. 9 shows measurement of tyrosinase protein expression in B16F1 melanoma cells by chrysophanol in dose-dependent manner; and

FIG. 10 schematically illustrates a skin whitening mechanism of chrysophanol.

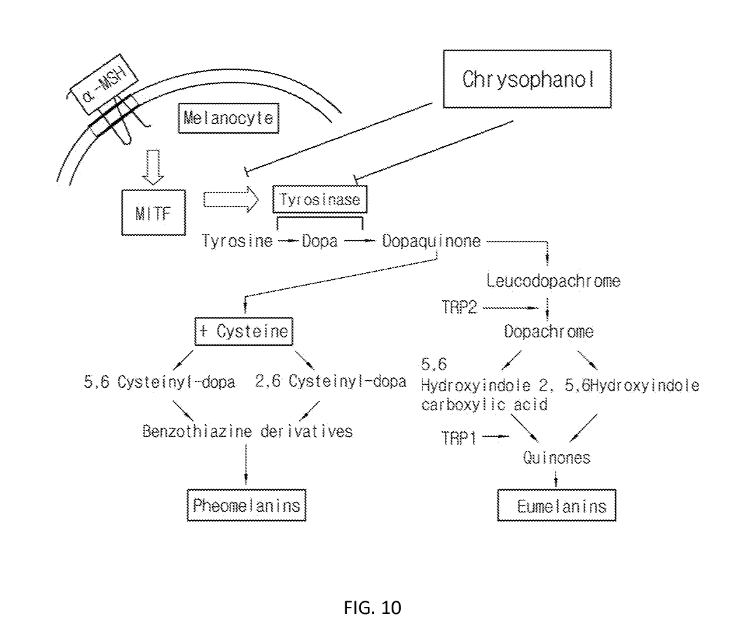

### DETAILED DESCRIPTION OF THE INVENTION

Hereinafter, the present invention will be explained in detail.

The present invention provides a cosmetic composition comprising chrysophanol represented by chemical formula I or pharmaceutically-acceptable salt thereof as an effective ingredient, for skin whitening:

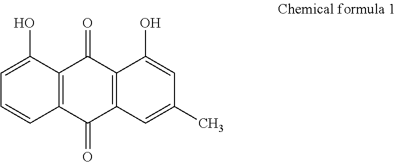 [SMILES: Cc1cc(O)c2c(c1)C(=O)c1cccc(O)c1C2=O]

Chrysophanol is anthraquinone derivative, and parietin is known to be extracted from a variety of plants including Rumex crispus and Rumex japonica, or produced by Aspergillus spp., Penicillium spp., and Alternaria spp. The molecular formula of chrisophanol compound is C₁₅H₁₀O₄ and molecular weight is 254.242.

Chrysophanol is known to have anticancer, antioxidant, and antimutation activity, and also known to possess topoisomerase inhibiting activity, toxicity to mouse when peritoneal injected, in vitro antiviral activity against poliovirus, and antibacterial activity against trichopyton mentagrophytes.

However, involvement with skin whitening effect has not been investigated so far.

While searching for a natural product derived substance which is stable in human body and which also provides excellent whitening effect, the present inventors have discovered for the first time that chrysophanol can effectively inhibit melanin synthesis by dose-dependently inhibiting expression and activity of tyrosinase, as a result of testing whitening effect with chrysophanol which was isolated from marine-derived fungus, Microsporum sp. and identified, and completed the present invention which provides a novel use of chrysophanol for skin whitening activity.

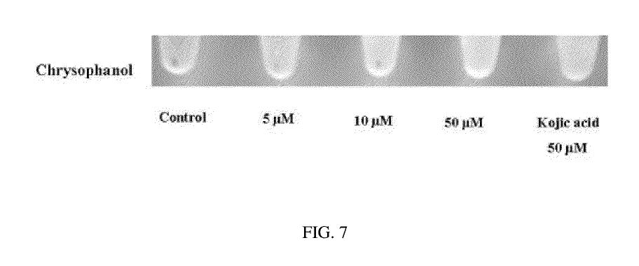

As a result of measuring tyrosinase inhibiting activity of chrysophanol compound in Experimental Example 2 explained below, it was confirmed that tyrosinase activity was inhibited by chrysophanol dose-dependently (see FIG. 7).

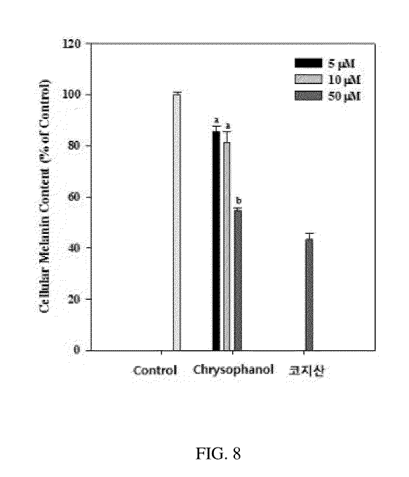

Further, as a result of treating B16F1 melanoma cells with chrysophanol compounds according to the present invention of different concentrations and inducing production of melanin with α-MSH, it was confirmed that melanin content was reduced in the B16F1 melanoma cell by the chrysophanol treatment dose-dependently. In a specific experiment group treated with 50 μM of chrysophanol, the melanin content was almost same as that of positive control (kojic acid), suggesting the excellent melanin production inhibiting activity of chrysophanol (FIGS. 7 and 8).

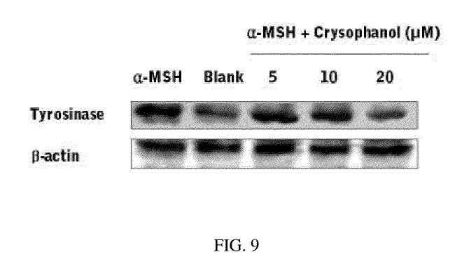

Further, referring to Experimental Example 4, as a result of treating different concentrations of chrysophanol compounds according to the present invention to B16F1 melanoma cells and inducing melanin production with α-MSH, it was observed through Western blot analysis that chrysophanol inhibited tyrosinase protein expression in B16F1 melanoma cell in dose-dependent manner (FIG. 9).

Collecting the results mentioned above, it was confirmed through experiments that chrysophanol is effective in skin whitening which is obtained by its activity to inhibit production of melanin.

According to the present invention, chrysophanol may be used in the form of salt, and preferably pharmaceutically-acceptable salt. The ‘salt’ as used herein may include acid-added salt formed by pharmaceutically-acceptable free acid. The free acid may use organic acid and inorganic acid. The organic acid may include Citric acid, acetic acid, lactic acid, tartaric acid, maleic acid, fumaric acid, formic acid, propionic acid, oxalic acid, trifluoroacetic acid, benzoic acid, gluconic acid, meta sulfonic acid, glycolic acid, succinic acid, 4-toluene sulfonic acid, glutamic acid, and aspartic acid, but not limited thereto. Further, the inorganic acid may include hydrochloric acid, bromic acid, sulfuric acid, and phosphoric acid, but not limited thereto.

According to the present invention, chrysophanol currently available on the market may be purchase, or isolated from a natural product, or prepared by a known chemical synthesis.

For example, chrysophanol according to the present invention may be extracted from various plants including Rumex crispus and Rumex japonica, and produced into second metabolites using Aspergillus spp., Penicillium spp., and Alternaria spp.

In one embodiment of the present invention, the chrysophanol may be derived from Microsporum sp.

When prepared into a form of cosmetic composition, the composition according to the present invention may include ingredients generally used in the cosmetic composition in addition to chrysophanol such as, for example, general adjuvant including antioxidant, stabilizer, solubilizer, vitamin, pigment and flavor, and carrier.

Further, in addition to the chrysophanol explained above, the composition according to the present invention may be mixed with an organic ultraviolet ray block agent traditionally used in the art to the extent that does not react with chrysophanol to counteract skin protection effect.

The organic ultraviolet ray blocking agent may include at least one selected from the group consisting of glyceril PABA, drometrizol trisiloxane, drometrizole, degalioyl trioleate, disodium phenyl dibenzimidazol tetrasulfonate, diethylhexyl butamido triazone, diethyl aminohydroxy benzoylhexylbenzoate, D-A-methoxycinnamate, compound of lawson and dihydroxyacetone, methylene bis-benzotriazolyl tetramethyl butylphenol, 4-methylbenzylidene camphor, menthyl anthranilate, benzophenon-3(oxybenzone), benzophenone-4, benzophenone-8 (dioxyphebenzone), butylmethoxy dibenzoylmethane, bis-ethylhexyl oxyphenol methoxy phenyltriazine, cynoxate, ethyldihydroxypropyl PABA, ethylhexyl methoxycinnamate, ethylhexylsalicylate, ethylhexyltraizone, isoamil-p-methoxycinate, polysilicon-15 (dimethycodiethylbenzalmalonate), terephthalylidene dicamphor sulfonic acid and salt thereof, TEA-salicylate and aminobenzoic acid (PABA).

Product that can contain the composition according to the present invention may include, for example, cosmetic products such as astringent, toner, nutrition, various creams, essence, pack, or foundation, and cleansing, cleanser, soap, treatment, or cosmetic fluid.

To be specific, a preparation of the cosmetic composition according to the present invention may include skin lotion, skin softener, skin toner, astringent, lotion, milk lotion, moisture lotion, nutrition lotion, massage cream, nutrition cream, moisture cream, hand cream, essence, nutrition essence, pack, soap, shampoo, cleansing foam, cleansing lotion, cleansing cream, body lotion, body cleanser, serum, lipstick, makeup base, foundation, pressed powder, loose powder, or eye shadow.

In one specific embodiment of the present invention, chrysophanol is contained in an amount of 0.0001-20 wt. %, preferably 0.5-20 wt % and more preferably, 1.0-20 wt % of the total weight of the composition. If the chrysophanol content is less than 0.0001 wt %, whitening effect by chrysophanol greatly decreases, while if the chrysophanol content excees 20 wt %, skin irritation can occur and problem associated with the preparation can also occur.

Further, in one specific embodiment of the present invention, chrysophanol may be contained in concentration of 10 to 50 μM of the composition. If the concentration of chyrsophanol is less than 10 μM, whitening effect by chrysophanol can be greatly deteriorated, while if chyrsophanol concentration exceeds 50 μM, cell toxicity is possible.

Meanwhile, a preparation of the cosmetic composition according to the present invention may contain chrysophanol in nanoliposome to ensure stability. When contained within nanoliposome, chrysophanol ingredient is stabilized and therefore, problems such as formation of sediment, discoloration or generation of foul odor during preparation, can be resolved, while dissolubility and percutaneous absorption increase. As a result, the maximum efficacy can be expected from the compound.

The ‘nanoliposome’ as used herein refers to a conventional form of liposome which has average diameter of particles ranging between 10-500 nm. In a preferred embodiment of the present invention, the average particle diameter of nanoliposome is between 50-300 nm. If the average particle diameter of the nanoliposome exceeds 300 nm, improvement of skin penetration and preparation safety, as intended to be achieved by the present invention, is relatively low.

The nanoliposome for use in stabilizing the compound (i.e., chrysophanol) may be prepared by a mixture of polyol, oil ingredient, surfactant, phospholipid, fatty acid and water.

Polyol for use in the nanoliposome according to the present invention is not particularly limited, but may preferably be one or more selected from the group consisting of propylene glycol, dipropylene glycol, 1,3-butyleneglycol, glycerin, methylpropandiol, isopropylene glycol, phenthylene glycol, erythritol, xylitol, sorbitol, and a mixture thereof. The polyol may be used in an amount of 10-80 wt %, and preferably, 30-70 wt % of the total weight of nanoliposome.

The oil ingredient for use in the preparation of the nanoliposome according to the present invention may include a variety of known oils, and preferably includes hydrocarbon oil such as hexadecane and paraffin oil, ester synthetic oil, silicone oil such as dimethicone and cyclomethicone, oils from animals and plants such as sunflower oil, corn oil, soybean oil, avocado oil, and sesame oil, sphingolipid such as ethoxylated alkylether oil, propoxylated alkylether oil, phytosphingosine, sphingosine and sphinganine, cerebroside cholesterol, sytosterol cholesteryl sulfate, sitosteryl sulfate, C₁₀₋₄₀ fatty alcohol and a mixture thereof. The oil ingredient may be used in an amount of 1.0-30.0 wt %, and preferably, 3.0-20.0 wt % of the total weight of the nanoliposome.

Any of the known surfactants may be used in the preparation of the nanoliposome according to the present invention. For example, anionic, cationic, or nonionic surfactant may be used. In a preferred embodiment, the anionic and the nonionic surfactant may be used. To be specific, the anionic surfactant may include alkylacyl glutamate, alkylphosphate, alkyl lactylate, dialkylphosphate and trialkylphosphate. The nonionic surfactant may specifically include alkoxylated alkylether, alkyoxylated alkylester, alkylpolyclycoside, polyglycerylester, and sugar ester. Particularly preferred surfactant is polysorbates belonging to nonionic surfactant family. The surfactant may preferably be used in an amount of 0.1-10 wt %, and preferably, 0.5-5.0 wt % of the total weight of the nanoliposome.

The phospholipid is also used in the preparation of nanoliposome according to the present invention and includes natural phospholipid (e.g., egg yolk lecithin, soybean lecithin, sphingomyelin) and synthetic phospholipid (e.g., dipalmitoylphosphatidylcholin or water-added lecithin), and preferably, lecithin. Naturally-derived unsaturated lecithin or saturated lecithin extracted from soybean or egg yolk is particularly preferred. Generally, naturally-derived lecithin includes 23-95% of phosphatidylcholine, and 20% or less phosphatidylethanolamine. In the preparation of the nanoliposome according to the present invention, phospholipid may be used in an amount of 0.5-20.0 wt %, and preferably, 2.0-8.0 wt % of the total weight of the nanoliposome.

The fatty acid used in the preparation of the nanoliposome according to the present invention is higher fatty acid, and preferably, C₁₂₋₂₂ alkyl chain saturated or unsaturated fatty acid, which includes, for example, lauric acid, myristic acid, palmitic acid, stearic acid, oleic acid and linoleic acid. The fatty acid may be used in an amount of 0.05-3.0 wt %, and preferably, 0.1-1.0 wt % of the total weight of the nanoliposome.

Water used in the preparation of the nanoliposome according to the present invention may generally be deionized distilled water and may be used in an amount of 5.0-40 wt % of the total weight of the nanoliposome.

The nanoliposome may be prepared by various known methods in the art, but most preferably, prepared by applying a mixture of the above-mentioned ingredients into a high pressure homogenizer. The preparation of the nanoliposome by the high pressure homogenizer may be performed under various conditions (e.g., pressure, number of times) depending on the targeted particle size, and preferably, the nanoliposom may be prepared by a process including 1-5 times of passing high pressure homogenizer under 600-1200 bar of pressure.

The cosmetic composition for skin improvement according to the present invention may include 0.1-20.0 wt % of chrysophanol of the total weight of the nanoliposome.

The present invention also provides a food composition for improvement skin whitening, comprising chrysophanol as an effective ingredient.

In addition to chrysophanol, the effective ingredient of the present invention, the food composition may include additional components such as flavoring agent or natural carbohydrate as other general food compositions do.

An example of the natural carbohydrate includes general sugar such as monosaccharide, for example, glucose, fructose, etc.: disaccharide, for example, maltose, sucrose etc.; and polysaccharide, for example, dextrin, cyclodextrin, and sugar alcohol such as xylitol, sorbitol, erythritol. The flavoring agent may advantageously use natural flavoring agent (thaumatin), stevia extract (e.g., rebaudioside A, glycyrrhizin, etc.) and synthetic flavoring agent (e.g., saccharine, aspartame, etc.).

The food composition according to the present invention may be formulated in the same manner as explained above with respect to the pharmaceutical composition and used as functional food, or added to various foods. The composition according to the present invention may be added to, for example, drinks, meat, chocolate, foods, snacks, pizza, ramyun, noodles, gums, candies, ice cream, alcoholic drinks, vitamin complex, and health supplementary foods, etc.

Further, in addition to chrysophanol, which is the effective ingredient, the food composition according to the present invention may contain various other components such as various nutrients, vitamin, mineral (electrolyte), flavoring agent including synthetic flavoring agent and natural flavoring agent, coloring agent and enhancer (e.g., cheese, chocolate, etc.), pectic acid and salt thereof, anginic acid and salt thereof, organic acid, protective colloidal thickener, pH regulator, stabilizer, preservative, glycerin, alcohol, carbonating agent used in carbonated beverages. Additionally, the food composition according to the present invention may include fruit flesh for preparation of natural fruit juice and fruit juice beverage and vegetable beverage.

Chrysophanol, the effective ingredient of the present invention, is derived from a natural substance, and thus is stable in human body and can be used for long period of time for skin whitening benefit.

The present invention also provides a health functional food for improvement of skin whitening, comprising chrysophanol as an effective ingredient.

The health functional food according to the present invention may be prepared in the form of tablets, capsules, powder, granules, liquid or pills for the purpose of skin whitening effect.

The term “health functional food” as used herein may refer to food manufactured and processed under rule No. 6727 related to the health functional food, using ingredient or component having effective functionalities to human bodies, and consumed for the purpose of effective benefits in terms of use for health such as regulation of nutrients or biological action regarding structure and functions of human body.

The health functional food according to the present invention may include general food additives, and unless specifically applied under other regulations, appropriateness of using a component as food additive is evaluated according to specification and standard of corresponding product category, based on the General Provisions and General Test Methods of Korea Food Additives Code (KFAC) approved by the Korea Food & Drug Administration.

The products listed in the KFAC includes, for example, chemical compounds such as ketones, glycin, calcium citrate, nicotinic acid, cinnamic acid, etc.; natural additives such as persimmon color, licorice extract, microcrystalline cellulose, Kaoliang color, guar gum, etc.: and mixture additives such as L-sodium glutamate, alkali agents for noodles, preservative, prepared tar dyes, etc.

For example, the health functional food in tablet dosage form may be made by a process of granulating a mixture of chrysophanol, the effective ingredient according to the present invention, with excipient, binder, disintegrant and other additives by the known method, and pressing the resultant mixture with an addition of glidant or the like, or directly pressing the mixture. The health functional food in tablet dosage form may include tasty acid depending on needs.

Among the capsule dosage forms, the health function food in hard capsule dosage form may be made by filling a general hard capsule with a mixture of chrysophanol, the effective ingredient according to the present invention, with additives such as excipient, or the like, and the health function food in soft capsule dosage form amy be made by filling a capsule material such as gelatin with a mixture of chrysophanol and additives such as expicient. The soft capsule may include plasticizer such as glycerin or sorbitol, colorant, preservative, or the like, depending on need.

The health functional food in pill dosage form may be made by shaping a mixture of chrysophanol, the effective ingredient according to the present invention, with excipient, binder, disintegrant, or the like by the known method, and depending on needs, may be coated with white sugar or other coaters, or coated on the surface thereof with a substance such as starch or talc.

The health functional food in granule dosage form may be clinically made by the known method with a mixture of chrysophanol, the effective ingredient according to the present invention, with excipient, binder, disintegrant, or the like, and may include fragrance ingredient or tasty acid as need arises.

The health functional food may be beverages, meat, chocolate, foods, snacks, pizza, ramyun, noodles, gums, candies, ice cream, alcoholic drinks, vitamin complex, and health supplementary foods, etc.

Hereafter, the present invention will be explained in detail with reference to examples and experimental examples. However, the examples are written only for illustrative purpose, and accordingly, the present invention is not limited to any specific examples.

### EXAMPLES

### Reagents

Mushroom tyrosinase, L-tyrosine, α-MSH (α-melanocyte stimulating hormone) and kojic acid were purchased from Sigma Chemical Company (St. Louis, Mo.). Antibody for tyrosinase (H0109) was purchased from Santa Cruz Biotechnology, Inc. (Santa Cruz, Calif.) and other reagents of analytical grades were used.

### Example 1

### Isolation and Culture of Marine-Derived Fungus Microsporum sp

The fungal strain (MFS-YL) was isolated from the surface of a marine red alga Lomentaria catenata, collected at Guryongpo, Nam-Gu, PoHang in Republic of Korea in 2009. The fungal strain was cultured in YPG medium (0.5% yeast extract, 0.5% peptone, 1% glucose, and 60% seawater) and preserved in 10% glycerol with the YPG medium. The further culture was preceded with the same medium (YPG, 1 L×20 Erlenmeyer flasks) to isolate secondary metabolites. The medium was sterilized (121° C., 15 lb/inch²) before the culture of fungal strain and inoculation was done after cool down the media under the laminar flow. The fungus was cultured for 30 days at 25° C. and identified as Microsporum sp. based on the cellular fatty acid composition with a similarity index of 0.62 by the Korean Culture Center of Microorganisms, Seoul, Republic of Korea.

### Example 2

### Extraction of Bioactive Secondary Metabolites from Marine-Derived Fungus Microsporum sp

The isolated fungal strain MFS-YL from Example 1 was cultured and the produced secondary metabolites were isolated/identified.

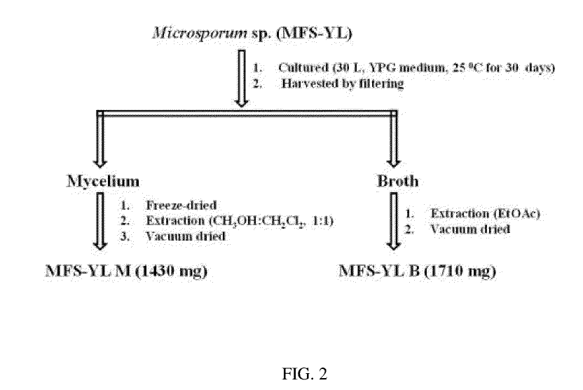

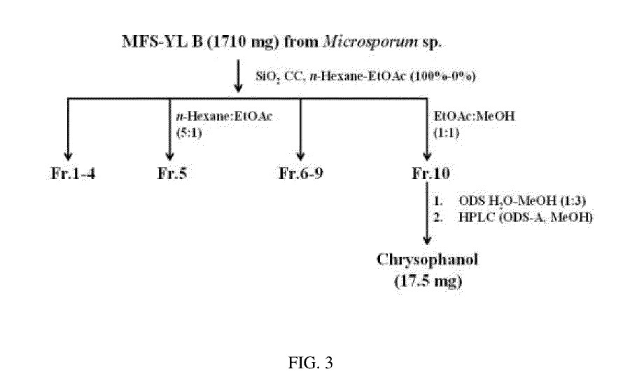

To be specific, the culture broth and mycelium were separated from the culture of Example 1 and the filtered broth was extracted with ethyl acetate (1:1.5 v/v, broth:EtOAc) and by silica gel flash chromatography (n-hexane:EtOAc chloroform:MeOH=1:1) and obtained ten fractions (F1-F10) from the ethyl acetate extract. Further purification was conducted by OOS column chromatography (water:EtOA), and high performance liquid chromatography (HPLC)(YMC OOS-A, MeOH) was conducted (see FIGS. 2 and 3).

### Example 3

### Identification of Compound

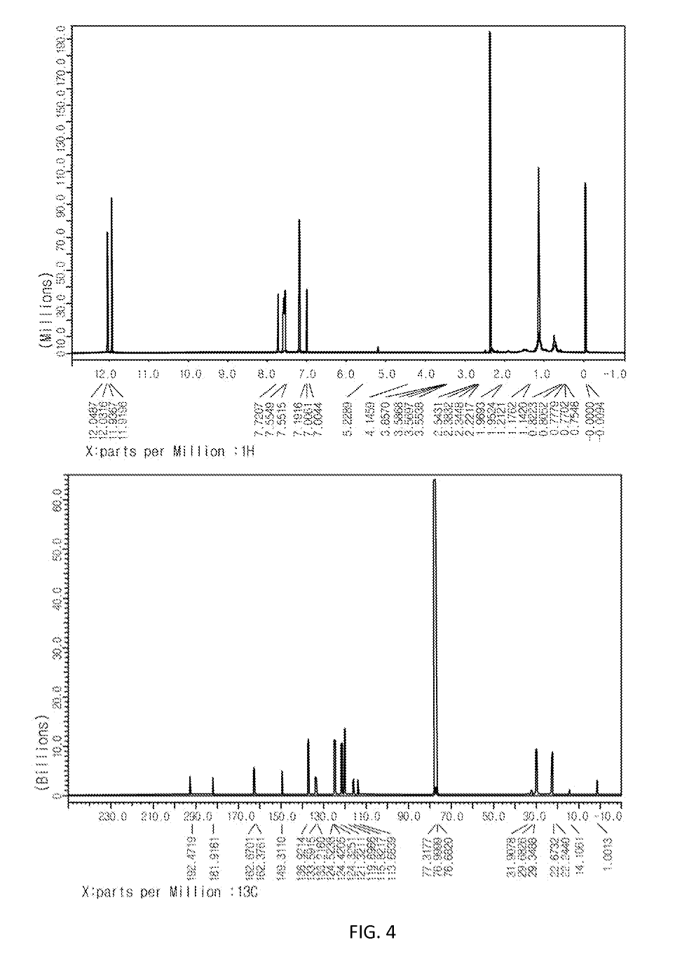

As a result of analyzing spectroscopic characteristics to identify the compound obtained from Example 2, the compound was identified to be chrysophanol (FIG. 4).

Chrysophanol, 17.5 mg: yellow crystalline compound: ¹H-NMR (CDCl₃, 400 MHz) δ 12.05 (1H, s, —OH), 11.93 (1H, s, —OH), 7.72 (1H, dd, J=8.4, 7.6 Hz, H-6), 7.61 (1H, d, J=7.6 Hz, H-5), 7.55 (1H, d, J=0.8 Hz, H-4), 7.22 (1H, d, J=8.4 Hz, H-7), 7.01 (1H, d, J=0.8 Hz, H-2), 2.34 (3H, s, —CH₃); ¹³C NMR (CDCl₃, 100 MHz) δ 192.5 (C-9), 181.9 (C-10), 162.7 (C-8), 162.4 (C-1), 149.3 (C-3), 136.9 (C-6), 133.6 (C-10a), 133.2 (C-4-a), 124.5 (C-2), 123.4 (C-7), 121.3 (C-4), 119.9 (C-5), 115.8 (C-8a), 113.7 (C-9a), 22.2 (—CH₃). LREIMS m/z: 254.11 [M]⁺ (C₁₅H₁₀O₄).

### Experimental Example 1

### Determination of Cytotoxicity by Chrysophanol

The mouse melanoma B16F1 cells were acquired from the American Type Culture Collection (ATCC, Manassas, Va.) and were cultured in Dulbecco's modification of eagle's medium (DMEM, GIBCO, New York, USA) containing 100 μg/ml penicillin-streptomycin, 10% μg/ml fetal bovine serum (FBS) and maintained at 37° C., 5% CO₂. Cytotoxicity levels of the isolated chrysophanol compound from Microsporum sp. on B16F1 cells were measured using MTT (3-(4,5-dimethyl-2-yl)-2,5-diphenyltetrazolium bromide) method.

Briefly, B16F1 cells were cultured in 96-well plates at a density of 5×10³ cells/well. After 24 h, cells were treated with different concentrations (10, 50, 100 μM) of chrysophanol compound. After the next 24 h of incubation, cells were washed two times with PBS and 100 μl of MTT solution (1 mg/ml) was added to each well. After next 4 h of incubation, MTT solution in each well was removed and then 100 μl of dimethyl-sulfoxide (DMSO) were added to solubilize the formazan salt. The optical density was measured at 540 nm by using UV microplate reader (Tecan Austria GmbH, Groedig, Austria). Relative cell viability was calculated compared to the non-treated blank group.

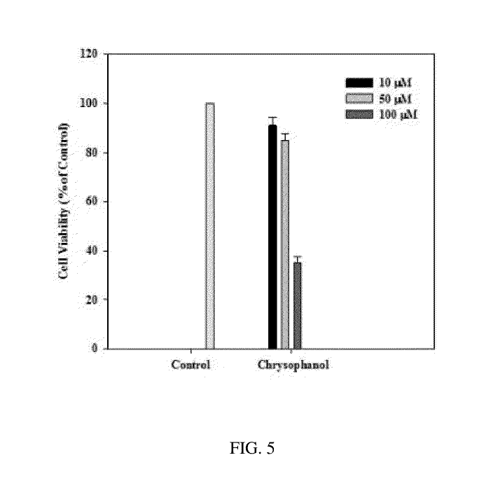

As a result, the experiment group treated with 10 and 50 μM concentrations of chrysophanol exhibit no cytotoxicity (FIG. 5). Accordingly, the appropriate concentration of chrysophanol for treatment is determined to be between 10 and 50 μM.

### Experimental Example 2

### Tyrosinase Inhibitory Activity of Chrysophanol

The inhibitory effect on tyrosinase was measured using the spectrophotometric method, based on the hydroxylation of L-DOOA in L-tyrosine in mushroom tyrosinase assay.

A total of 10 μl of each chrysophanol with different concentrations and 20 μl of mushroom tyrosinase (1000 units/ml) in 50 mM phosphate buffer (pH 6.5) were added to 170 μl of an assay mixture containing a ratio of 10:10:9 of 1 mM L-tyrosine solution, 50 mM phosphate buffer (pH 6.5), and distilled water in a 96-well microplate. After 30 min of incubation at 37° C., the absorbance of the mixture was determined at 490 nm using a microplate reader (Tecan Austria GmbH, Austria).

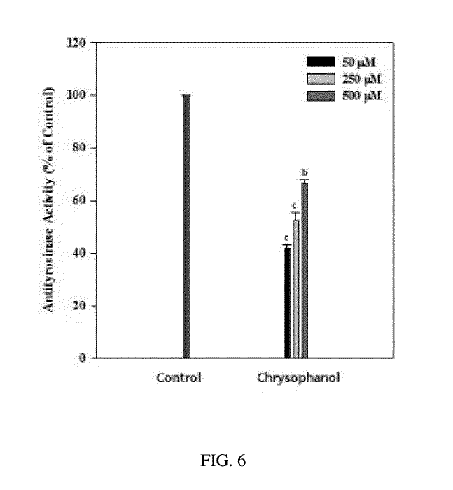

As a result, tyrosinase inhibitory activity was observed by chrysophanol in a dose-dependent manner (FIG. 6).

### Experimental Example 3

### Melanin Production Inhibitory Effect of Chrysophanol

The melanin content was determined in accordance a procedure described previously (Hosoi et al., 1985) with slight modifications.

Briefly, B16F1 cells were seeded at a density of 2×10⁴ cells per well in 24-well culture plates and then incubated for 24 h. Then, cells were treated with various concentrations (5, 10, 50 μm) of chrysophanol. After 1 h, α-MSH (100 nM) was added and incubated for 72 h. Then, cells were washed twice with PBS and dissolved in 1 N NaOH by 30 min of boiling at 60° C. The cell lysates were centrifuged for 5 min at 5000 g and absorbance of the supernatant was measured at 405 nm.

As a result, decrease in melanin content in B16F1 cells by the treatment of chrysphanol in a dose-dependent manner was observed (FIGS. 7 and 8), and particularly, the melanin content of the experiment group treated with 50 μM was almost the same as that of kojic acid, the positive control, thereby suggesting the excellent melanin production inhibitory effect of chrysophanol.

### Experimental Example 4

### Inhibitory Effect of Chrysophanol on Expression Level of Tyrosinase Protein

As mentioned above in Experiment Example 2, it was revealed that chrysophanol can inhibit tyrosinase activity, and to investigate the inhibitory effect of chrysophanol on expression of tyrosinase protein in mouse melanoma B16F1 cells, the expression level of tyrosinase protein according to the treatment with chrysophanol was investigated by western blot analysis.

For the western blot analysis, B16F1 cells were treated with different concentrations of chrysophanol compound, added with α-MSH, incubated for a predetermined time, and then the cells were lysed with RIPA buffer (Sigma-Aldrich Corp., St. Louis, USA) and centrifuged. After the centrifugation, the same amount (15 μg) of protein in cell lysate supernatants were analyzed on 10% SDS-PAGE and proteins were blotted on an immuno-blot nitro-cellulose membrane and blocked with 5% BSA in TBS containing 0.1% Tween 20 (TBS-T) for at least 1 h and hybridized with the primary monoclonal antibodies (Santa Cruz Biotechnology Inc., CA, USA). All the primary monoclonal antibodies were diluted with TBS-T (1:1000). The protein bindings were detected using the secondary antibody conjugated to horseradish peroxidase and enhanced using chemiluminiscence ECL assay kit (Amersham Pharmacia Biosciences, England, UK) according to the manufacturer's instructions and imaged on a LAS-3000® Luminescent image analyzer (Fujifilm, Life Science, Tokyo, Japan). T

As a result, dose-dependent inhibition of tyrosinase protein expression was observed in the B16F1 cells by the treatment with chrysophanol, and particularly, the experiment group treated with 20 μM of chrysophanol exhibited significantly inhibited expression of tyrosinase protein (FIG. 9).

Accordingly, it is determined that, by the mechanism of inhibiting activity and expression of tyrosinase, the chrysophanol compound derived from marine-derived fungus can effectively inhibit the melanin synthesis, and thus can be advantageously used as a skin whitening substance.

The foregoing embodiments and advantages are merely exemplary and are not to be construed as limiting the present invention. The present teaching can be readily applied to other types of apparatuses. Also, the description of the exemplary embodiments of the present inventive concept is intended to be illustrative, and not to limit the scope of the claims, and many alternatives, modifications, and variations will be apparent to those skilled in the art.

What is claimed is:

1. A cosmetic composition for skin whitening, comprising chrysophanol represented by Chemical formula 1 or pharmaceutically-acceptable salt thereof:

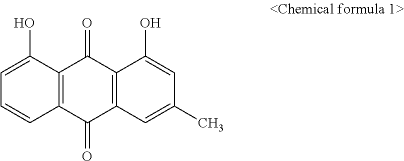 [SMILES: Cc1cc(O)c2c(c1)C(=O)c1cccc(O)c1C2=O]

2. The cosmetic composition as set forth in claim 1, wherein the chrysophanol is derived from marine-derived fungus, Microsporum sp.

3. The cosmetic composition as set forth in claim 1, wherein the chrysophanol inhibits production of melanin via mechanism of inhibiting expression and activity of tyrosinase.

4. The cosmetic composition as set forth in claim 1, wherein the chrysophanol is included in the composition in a concentration of 10 to 50 μM.

5. The cosmetic composition as set forth in claim 1, wherein the composition is formulated into one selected from the group consisting of: skin lotion, skin softener, skin toner, astringent, lotion, milk lotion, moisture lotion, nutrition lotion, massage cream, nutrition cream, moisture cream, hand cream, essence, nutrition essence, pack, soap, shampoo, cleansing foam, cleansing lotion, cleansing cream, body lotion, body cleanser, serum, lipstick, makeup base, foundation, pressed powder, and loose powder.

6. The cosmetic composition as set forth in claim 2, wherein the composition is formulated into one selected from the group consisting of: skin lotion, skin softener, skin toner, astringent, lotion, milk lotion, moisture lotion, nutrition lotion, massage cream, nutrition cream, moisture cream, hand cream, essence, nutrition essence, pack, soap, shampoo, cleansing foam, cleansing lotion, cleansing cream, body lotion, body cleanser, serum, lipstick, makeup base, foundation, pressed powder, and loose powder.

7. The cosmetic composition as set forth in claim 3, wherein the composition is formulated into one selected from the group consisting of: skin lotion, skin softener, skin toner, astringent, lotion, milk lotion, moisture lotion, nutrition lotion, massage cream, nutrition cream, moisture cream, hand cream, essence, nutrition essence, pack, soap, shampoo, cleansing foam, cleansing lotion, cleansing cream, body lotion, body cleanser, serum, lipstick, makeup base, foundation, pressed powder, and loose powder.

8. The cosmetic composition as set forth in claim 4, wherein the composition is formulated into one selected from the group consisting of: skin lotion, skin softener, skin toner, astringent, lotion, milk lotion, moisture lotion, nutrition lotion, massage cream, nutrition cream, moisture cream, hand cream, essence, nutrition essence, pack, soap, shampoo, cleansing foam, cleansing lotion, cleansing cream, body lotion, body cleanser, serum, lipstick, makeup base, foundation, pressed powder, and loose powder.
